(19) United States
(12) Patent Application Publication
(10) Pub. No.: US 2003/0022158 A1
(43) Pub. Date: Jan. 30, 2003

(54) Peptides from the TT virus sequence and monospecific antibodies binding to the TT virus

(76) Inventor: Matti Sallberg, Alvsjo (SE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/992,896

(22) Filed: Nov. 5, 2001

Related U.S. Application Data
(63) Continuation-in-part of application No. PCT/EP00/03958, filed on May 3, 2000.

(30) Foreign Application Priority Data
May 4, 1999 (SE) SE19990001601

(51) Int. Cl. C12Q 1/70; C07H 21/04; A61K 39/395; A61K 39/12; C07K 2/00; C07K 4/00; C07K 5/00; C07K 7/00; C07K 14/00; C07K 16/00; C07K 17/00; A61K 38/00; C12N 5/06; C12N 5/16; C07K 1/00; G01N 33/53; G01N 33/537; G01N 33/543

(52) U.S. Cl. 435/5; 435/345; 530/300; 530/350; 536/23.72; 424/130.1; 424/204.1; 435/7.1; 435/7.95

(57) ABSTRACT

The present invention relates to peptides derived from the genomic sequence of the TT virus and antibodies generated therefrom, which can be used in diagnostics and medicaments for the diagnosis, treatment, and prevention of TT virus infection. Several synthetic peptides generated according to the methods described herein were found to be reactive to antibodies present in serum from patients suffering from TT virus infection. A peptide having the sequence of SEQ ID NO: 1 was introduced into mammals and was found to induce the production of antibodies specific for said peptide.

### CROSS-REFERENCE TO RELATED PATENTS

This application is a continuation-in-part of international application number PCT/EP00/03958 and claims the benefit of priority of international application number PCT/EP00/03958 having international filing date of May 3, 2000, designating the United States of America and published in English, which claims the benefit of priority of Swedish patent application No. SE19990001601, filed May 4, 1999; both of which are hereby expressly incorporated by reference in their entireties.

### FIELD OF THE INVENTION

The present invention relates to peptides derived from the genomic sequence of the TT virus. More specifically, embodiments of the invention include peptides that correspond to regions of the TT virus and antibodies generated therefrom, which can be used in diagnostics and medicaments for the diagnosis, treatment, and prevention of TT virus infection.

### BACKGROUND OF THE INVENTION

In 1997, a new human infectious agent, later named the TT virus (TTV), was identified from the serum of a Japanese patient with post transfusion non-A-G hepatitis. (Nishizawa et al., Biochem Biophys Res Commun, 241:927 (1997)). TTV DNA was detected in 47% of patients with fulminate non-A-G hepatitis and 46% of patients with chronic liver disease of unknown etiology (Okamoto et al., J Med Virol, 56:128-32 (1998)). TTV is now recognized as a global problem, (Cossart, Lancet, 352:164 (1998)) and appears to be more common in populations with increased risk for infection by blood borne viruses (Okamoto et al., J Med Virol, 56:128-32 (1998)), such as, populations with high concentrations of hemophiliacs and drug addicts. Non-parenteral transmission of TT virus is also possible, however. (Okamoto et al., J Med Virol, 56:128-32 (1998)).

TTV is a non-enveloped, single-stranded DNA virus with a genome of at least 3,7 kb. (Okamoto et al., J Med Virol, 57:252-8 (1999)). It has a range of sequence divergence, allowing classification into different genotypes and subtypes (Okamoto et al., J Med Virol, 57:252-8 (1999)). A relationship with the family Parvoviridae has been hypothesized. (Okamoto et al., J Med Virol, 57:252-8 (1999)). Current research has revealed evidence of hepatotropism (Okamoto et al., J Med Virol, 56:128-32 (1998)) and, in some patients with non-A-G post-transfusion hepatitis and TTV viremia, TTV DNA titres were correlated with aminotransferase levels. (Nishizawa et al., Biochem Biophys Res Commun, 241:92-7 (1997)).

There has been little evidence to support an association between TTV infection and severe liver disease, however. (Cossart, Lancet, 352:164 (1998); Naoumov et al., Lancet, 352:195-7 (1998); Viazov et al., J Clin Virol, 11:183-7 (1998)). The epidemiological, immunological, and clinical significance of TTV infection remains uncertain. Moreover, serological tests for TTV infection are not yet available and, at the moment, PCR is the only available diagnostic tool. The need for diagnostics to identify TTV infection and medicaments to treat and/or prevent this disease is manifest.

### SUMMARY OF THE INVENTION

The present invention concerns the discovery that peptides encoded by different regions of the genomic sequence from the TT virus (TTV) can generate antibodies that are specific for TTV. Serum samples obtained from patients with TTV infection were found to be reactive with a peptide of the sequence: TATTTTYAYPGTNRPPV (SEQ ID NO: 1) and deletion mapping of this peptide revealed that the human antibodies present in the serum from infected individuals were directed to the sequence: YAYPGTNRPPV (SEQ ID NO: 2).

Embodiments of the invention concern an isolated or purified peptide consisting of, consisting essentially of, or comprising an amino acid sequence selected from the group consisting of: SEQ ID NO: 1, SEQ ID NO:2, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 60, SEQ ID NO: 61, SEQ ID NO: 62, SEQ ID NO: 63, and SEQ ID NO: 64. Each of these peptides are also individually embodiments of the invention.

Embodiments of the invention also concern mixtures of the peptides above. For example, one mixture includes the peptide: TATTTTYAYPGTNRPPV (SEQ ID NO: 1) or a peptide of SEQ ID NO: 1, wherein one to all six of the N-terminal amino acids are omitted, and a peptide of a sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19. The peptides described above can be coupled or joined to a carrier or label or can be immobilized on a solid phase. The peptides of the invention can be used for diagnostic purposes, for example, to identify the presence or absence of TTV in a subject or as biological tools, for example, to isolate human antibodies specific for TTV. The peptides described herein are preferably used as immunogens to generate antibodies in a mammal (e.g., as a vaccination or immunization protocol).

Embodiments of the invention also include isolated or purified antibodies specific for the peptides described herein and, thus, TTV. These antibodies can be monoclonal or polyclonal and are, preferably, monospecific. Medicaments (e.g., vaccines) containing said antibodies and, optionally, an adjuvant can be administered to a subject in need to treat and/or prevent TTV infection.

Diagnostic kits containing the peptides and/or the antibodies described above are also embodiments. These kits can also, optionally, have detection reagents, buffers, positive and negative controls, enzymes, and instructions. Preferred kits involve dipstick assays whereby one can detect the presence or absence of TTV in a sample using a dipstick having a peptide or antibody described herein joined to said dipstick.

Methods of diagnosing the presence or absence of TTV in a subject using the embodied peptides or antibodies are also embodiments, as well as, methods of using the disclosed peptides to generate an immune response (e.g., antibody production) in a recipient mammal. Moreover, embodiments include methods of making medicaments for the treatment and/or prevention of TTV infection whereby the disclosed peptides or antibodies are formulated into a pharmaceutical form.

### DETAILED DESCRIPTION OF THE INVENTION

The present invention concerns the discovery that peptides encoded from specific regions of the genomic sequence of the TT virus (TTV) can be used to generate antibodies that are specific for the TT virus. A total of 80 overlapping peptides corresponding to the two open reading frames of TTV—ORFs 1 and 2—(Genebank accession no. AB008394) were synthesized. These peptides were screened against eight human serum samples from patients with TTV infection and eight human serum samples from patients without TTV infection to determine if antibodies in said samples were reactive to the specific peptides tested. Every sample from patients with TTV infection was found to be reactive with a peptide of the sequence: TATTTTYAYPGTNRPPV (SEQ ID NO: 1). Deletion and substitution mapping of this peptide was conducted and the reactivity of the samples were mapped to the sequence: YAYPGTNRPPV (SEQ ID NO: 2), wherein the residues “PV” were found to be significantly involved in the binding of human antibodies.

Accordingly, embodiments of the invention concern an isolated or purified peptide consisting of, consisting essentially of, or comprising the amino acid sequence: TATTTTYAYPGTNRPPV (SEQ ID NO: 1). In some embodiments, 1, 2, 3, 4, 5, or 6 of the N-terminal amino acids of SEQ ID NO: 1, that is, TATTTT (SEQ ID NO: 3), can be omitted. For example, embodiments include isolated or purified peptides that consist of, consist essentially of, or comprise a peptide of the amino acid sequence: YAYPGTNRPPV (SEQ ID NO: 2), TYAYPGTNRPPV (SEQ ID NO: 60), TTYAYPGTNRPPV (SEQ ID NO: 61), TTTYAYPGTNRPPV (SEQ ID NO: 62), TTTTYAYPGTNRPPV (SEQ ID NO: 63), and ATTTTYAYPGTNRPPV (SEQ ID NO: 64).

The term “isolated” requires that the material be removed from its original environment (e.g., the natural environment if it is naturally occurring). That is, a naturally occurring protein or peptide present in a living cell is not isolated, but the same protein or peptide, separated from some or all of the coexisting materials in the natural system, is isolated. For example, an antibody from human serum is considered to be isolated when the antibody is removed from other components present in serum, such as, when said antibody remains bound to an immobilized antigen after a wash in a suitable buffer. The term “purified” does not require absolute purity; rather it is intended as a relative definition. For example, proteins and peptides are routinely purified to electrophoretic homogeneity, as detected by Coomassie staining, and are suitable in several assays despite having the presence of contaminants.

Aspects of the invention are also directed to an isolated or purified peptide mixture consisting of, consisting essentially of, or comprising the peptide: TATTTTYAYPGTNRPPV (SEQ ID NO: 1) or a peptide of SEQ ID NO: 1, wherein one to all six of the N-terminal amino acids are omitted, and a peptide of a sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, and SEQ ID NO: 19.

The peptides described above and/or at least one of the peptides in the peptide mixture described above, optionally, can be coupled to a carrier and/or a label. Examples of carriers are plastic surfaces, such as microplates, beads, etc.; organic molecules such as biotin; proteins, such as bovine serum albumin; peptide linkers, or polypeptides. Examples of labels that can be used, primarily for diagnostic purposes, are radioactive isotopes, enzymes, fluorescent markers, etc. Further, the peptides described above and/or at least one of the peptides in the peptide mixture described above can be immobilized on a solid phase, such as a glass or plastic surfaces, primarily for diagnostic purposes or purification of antibodies.

The peptides and peptide mixtures described above can be used for diagnostics and therapeutic purposes (e.g., to diagnose the presence of TTV infection in an individual or to raise antibodies to TTV in an inoculated individual). In either the diagnostics or therapeutics embodied herein, the peptides, optionally, can be coupled to or combined with other biologically active or inactive ingredients, such as carriers, adjuvants, and exipients.

Embodiments of the invention also concern isolated or purified antibodies that specifically bind TTV. Depending on the context, the term “antibodies” can encompass polyclonal, monoclonal, chimeric, single chain, Fab fragments and fragments produced by a Fab expression library. Preferably, the antibodies are monospecific. These antibodies can be used in diagnostics and medicaments, and can be, optionally, coupled to or combined with other biologically active or inactive ingredients, such as carriers, adjuvants, and exipients.. Some embodiments, for example, concern isolated or purified antibodies that specifically bind to a peptide or protein comprising, consisting essentially of, or consisting of an amino acid sequence selected from the group consisting of the amino acid sequences: SEQ ID NO: 1, SEQ. ID NO: 2, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 60, SEQ ID NO: 61, SEQ ID NO: 62, SEQ ID NO: 63, and SEQ ID NO: 64.

Embodiments of the invention also include diagnostic kits that comprise a peptide or peptide mixture, as described above, and, optionally, can contain reagents such as buffers, enzymes, positive and negative controls, instructions, and detection reagents. The kits can be used for immunological detection of TTV in biological samples, for example by using EIA, RIA, and dipstick assays, to detect the presence of antibodies against TTV in a biological fluid, such as blood or plasma. Embodiments of the invention also include diagnostic kits that comprise one or more antibodies, as described above, and, optionally, can contain reagents such as buffers, enzymes, positive and negative controls, instructions, and detection reagents. The kits can be used for immunological detection of TT virus in biological samples, for example by using EIA, RIA, and dipstick assays to detect the presence of antibodies against TTV in a biological fluid, such as blood or plasma. The diagnostic kits preferably contain the additional ingredients above, however, the inclusion of these additional ingredients will depend on the actual assay to be used with the specific kit.

Embodiments of the invention also concern methods of diagnosing the presence or absence of TTV in a subject. Some diagnostic methods are practiced, for example, by identifying a subject in need of a diagnosis of the presence or absence of TTV, obtaining a biological sample from said subject (e.g., a serum sample), contacting said sample with an isolated or purified peptide described herein (e.g., a peptide comprising, consisting essentially of, or consisting of an amino acid sequence selected from the group consisting of the amino acid sequences: SEQ ID NO: 1, SEQ. ID NO: 2, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 60, SEQ ID NO: 61, SEQ ID NO: 62, SEQ ID NO: 63, and SEQ ID NO: 64), and detecting the presence or absence of antibodies to said peptide in said sample.

Other methods of diagnosing the presence or absence of TTV in a subject involve identifying a subject in need of a diagnosis of the presence or absence of TTV, obtaining a biological sample from said subject (e.g., a blood sample), contacting said sample with an isolated or purified antibody (preferably a monoclonal antibody) directed to a peptide described herein (e.g., a peptide comprising, consisting essentially of, or consisting of an amino acid sequence selected from the group consisting of the amino acid sequences: SEQ ID NO: 1, SEQ. ID NO: 2, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 60, SEQ ID NO: 61, SEQ ID NO: 62, SEQ ID NO: 63, and SEQ ID NO: 64), and detecting the presence or absence of antigen in said sample.

Aspects of the invention also concern the use of the isolated or purified peptides or peptide mixtures described herein as immunogens for the purpose of stimulating a recipient mammal, including a human, to generate antibodies to TTV. Accordingly, some methods involve identifying a subject in need of antibodies to TTV, contacting said subject with an amount of isolated or purified peptide described herein (e.g., a peptide comprising, consisting essentially of, or consisting of an amino acid sequence selected from the group consisting of the amino acid sequences: SEQ ID NO: 1, SEQ. ID NO: 2, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 60, SEQ ID NO: 61, SEQ ID NO: 62, SEQ ID NO: 63, and SEQ ID NO: 64), that is sufficient to generate an immune response in said subject, and, optionally, measuring the antibody or immune response from said subject.

More embodiments concern methods of making medicaments for the treatment and/or prevention of TTV infection. By one approach, an isolated or purified peptide comprising, consisting essentially of, or consisting of an amino acid sequence selected from the group consisting of the amino acid sequences: SEQ ID NO: 1, SEQ. ID NO: 2, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 60, SEQ ID NO: 61, SEQ ID NO: 62, SEQ ID NO: 63, and SEQ ID NO: 64 is provided and said peptide is formulated into said medicament with or without a carrier. The section below describes the PCR methods that were employed to identify patients suffering from TTV.

Identification of patients suffering from TTV using PCR

By experimental design, several human serum samples were first screened using PCR to identify individuals that were infected with TTV. Once TTV positive individuals were identified using PCR, serum obtained from the TTV infected individuals was used to identify specific peptides that react with the anti-TTV antibodies present in infected patient's serum. Thus, the PCR screening step was employed to identify patients whose serum could be used in later experiments to identify reactive peptides.

To conduct the PCR screening, serum samples were obtained from a serum bank containing healthy blood donors, children with or without liver disease, mothers with IVDU (intravenous drug use) and their children. Total DNA was isolated from 50 μl of patient serum by phenol/chloroform purification. The DNA of all the patients was analyzed with two different degenerative primer sets, using (semi) nested PCR. Five μl of patient DNA were added to a 45 μl reaction mix containing 1 U of Taq polymerase (Perkin-Elmer applied Biosystems, Norwalk, Colo.), 10×PCR buffer, 200 μmol MgCl₂, dNTPs (125 μmol/nucleotide) and 20 pmol of each primer. The first round of synthesis upstream primer was: 5TTVout5 (5′-ACA GAC AGA GGA GAA GGC AAC ATG-3′) (SEQ ID NO: 4) and the downstream primer was either: 3TTVout (5′-CTG GCA TTT TAC CAT TTC CAA AGT T-3′) (SEQ ID NO: 5) or 3TTXout (5′-TAC CAY TTA GCT CTC ATT CTW AT-3′) (SEQ ID NO: 6). The DNA was amplified as follows: 95° C for 4.5 minutes, 32 cycles of 95° C. for 30 sec, 50° C. for 30 sec and 72° C. for 1 min, and one cycle of 95° C. for 30 sec, and 72° C. for 4 min.

A second round of PCR was performed using 5 μl of the first round PCR product under identical conditions. The second round of synthesis inner primers (upstream) were either 5TTVin (5′-GGC AAC ATG YTR TGG ATA GAC TGG-3′) (SEQ ID NO: 7) or 5TTVXin (5′-ACA GGA GAC HMA AAC ATA SA-3′) (SEQ ID NO: 8) and the down stream primer was 3TTVout (5′-CTG GCA TTT TAC CAT TTC CAA AGT T-3′) (SEQ ID NO: 5). The expected size of amplification products (275 bp for the first round and 140 bp for the second) was observed in agarose gel electrophoresis (3%). Samples that were either positive with both primer sets or reproducibly positive with one primer set were considered to be TTV positive. The primers were designed from the publicly available TTV sequence (Genebank accession no AB008394, herein expressly incorporated by reference in its entirety). See also Okamoto et al., Hepatol. Res. 10: 1-16 (1998), herein expressly incorporated by reference in its entirety. Once patients having TTV were identified using PCR, peptides corresponding to various regions of the TTV genomic sequence were prepared and analyzed. The section below describes the synthesis of peptides corresponding to various regions of the TTV sequence and the identification of specific peptides that interact with antibodies present in TTV infected patient serum.

Synthesis of TTV peptides and their use to detect human antibodies directed to TTV

Overlapping peptides (18 amino acids in length containing an 8 amino acid overlap) corresponding to regions of the ORFI and ORF2 of TTV (TABLE 1; Genebank accession no AB008394) were produced by a multiple peptide synthesizer (Syro, Syntex, Germany) using standard Fmoc chemistry (Sallberg et al., Immunology Letters, 30:59 68 (1991), herein expressly incorporated by reference in its entirety).

TABLE 1

| | |
|---|---|
| Complete amino acid sequences of the ORFs 1 and 2 of TTV* | |
| ORF1 | |
| MAYGWWRRRRRRWRRWRRRPWRRRWRTRRRRPARRRGRRRNVRRRRRGGRWRR | (SEQ ID NO:9) |
| RYRRWKRKGRRRKKAKIIIRQWQPNYRRRCNIVGYIPVLICGENTVSRNYATHSDDT | |
| NYPGPFGGGMTTDKFTLRILYDEYKRFMNYWTASNEDLDLCRYLGVNLYFFRHPDV | |
| DFIIKINTMPPFLDTELTAPSIHPGMLALDKRARWIPSLKSRPGKKHYIKIRVGAPRMFT | |
| DKWYPQTDLCDMVLLTVYATAADMQYPFGSPLTDSVVVNFQVLQSMYDKTISILPD | |
| EKSQREILLNKILASYIPFYNTTQTIAQLKPFIDAGNVTSGATATTWASYINTTKFTTATT | |
| TTYAYPGTNRPPVTMLTCNDSWYRGTVYNTQIQQLPIKAAKLYLEATKTLLGNTFTN | |
| EDYTLEYHGGLYSSIWLSPGRSYFETTGAYTDIKYNPFTDRGEGNMLWIDWLSKKN | |
| MNYDKVQSKCLISDLPLWAAAYGYVEFCAKSTGDQNIHMNARLLIRSPFTDPQLLVH | |
| TDPTKGFVPYSLNFGNGKMPGGSSNVPIRMRAKWYPTLFHQQEVLEALAQSGPFAY | |
| HSDIKKVSLGMKYRFKWIWGGNPVRQQVVRNPCKETHSSGNRVPRSLQIVDPKYNS | |
| PELTFHTWDFRRGLFGPKAIQRMQQQPTTTDIFSAGRKRPRRDTEVYHSSQEGEQKES | |
| LLFPPVKLLRRVPPWEDSQQEESGSQSSEEETQTVSQQLKQQLQQQRILGVKLRLLFN | |
| QVQKIQQNQD INPTLLPR GGDLASLFQIAP | |
| ORF2 | |
| MAEFSTPVRSGEATEGDLRVPRAGAEGEFTHRSQGAIRARDWPGYGQGSEKSMFIGR | (SEQ ID NO:10) |
| HYRKKRALSLCAVRTTKKACKLLIVMWTPPRNDQHYLNWQWYSSILSSHAAMCGC | |
| PDAVAHFNHLASVLRAPQNPPPPGPQRNLPLRRLPALPAAPEAPGDRAPWPMAGGAE | |
| GEDGGAGGDADHGGAAGGPEDADLLDAVAAAE | |

*(Genebank accession no. AB008394) used for the synthesis of the 80 overlapping peptides.

The EIAs used to identify which peptides interacted with human sera followed protocols known in the art. (See Zhang et al., J Gen Virol, 78:2735-2746 (1997), herein expressly incorporated by reference in its entirety). Briefly, microplates (Nunc, Denmark) were coated for 48 hours with synthetic peptides at a concentration of 10 μg/ml in 0.05M sodium carbonate buffer pH 9.6. After blocking for 2 hours at room temperature with phosphate buffered saline containing 1% bovine serum albumin, 2% goat serum and 0.05% Tween 20 (dilution buffer) the plates were incubated with human sera diluted 1:100 in dilution buffer. Bound human IgG was detected by incubation with anti-human IgG antibodies conjugated to alkaline phosphatase (Sigma Chemicals, St. Louis, Mo.). The plates were developed by the addition of dinitro-phenylene-diamine (Sigma) and the optical densities were determined at 405 nm.

Human reactivities to the 97 peptides derived from ORF1 and ORF2 are provided in TABLES 2 and 3. Reactive peptides within ORFI were found to be: peptides 10 (SEQ ID NO: 11), 18 (SEQ ID NO: 12), 29 (SEQ ID NO: 13), 35 (SEQ ID NO: 1), 42 (SEQ ID NO: 14), 44 (SEQ ID NO: 15), 50 (SEQ ID NO: 16), 51 (SEQ ID NO: 17), and 69 (SEQ ID NO: 18) (TABLE 2). Two of the tested human sera were reactive with peptide 19 (SEQ ID NO: 19) from ORF2 (TABLE 3). All reactive peptides are listed in TABLE 4.

TABLE 2
Analysis of human antibody reactivities in EIA to overlapping synthetic peptides corresponding to the complete open
reading frame 1 (ORF1) of the TT virus*

| TTV | | | | | | | | | | | | | | | | |
|---|---|---|---|---|---|---|---|---|---|---|---|---|---|---|---|---|
| ORF1 | Samples negative for TTV DNA by PCR | | | | | | | | Samples positive for TTV DNA by PCR | | | | | | | |
| peptide | 1 | 2 | 3 | 4 | 5 | 6 | 7 | 11 | 9 | 18 | 36 | 48 | 98 | 110 | 155 | 157 |
| 1 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 2 | 0.08 | 0.08 | 0.07 | 0.11 | 0.07 | 0.09 | 0.08 | 0.14 | 0.07 | 0.06 | 0.08 | 0.10 | 0.05 | 0.08 | 0.07 | 0.08 |
| 3 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 4 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 5 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 6 | 0.08 | 0.08 | 0.07 | 0.10 | 0.07 | 0.09 | 0.07 | 0.11 | 0.08 | 0.06 | 0.08 | 0.09 | 0.05 | 0.07 | 0.08 | 0.07 |
| 7 | 0.07 | 0.07 | 0.08 | 0.10 | 0.07 | 0.08 | 0.08 | 0.10 | 0.07 | 0.064 | 0.07 | 0.09 | 0.05 | 0.07 | 0.08 | 0.08 |
| 8 | 0.07 | 0.06 | 0.07 | 0.08 | 0.07 | 0.09 | 0.07 | 0.10 | 0.07 | 0.07 | 0.08 | 0.10 | 0.05 | 0.07 | 0.09 | 0.09 |
| 9 | 0.07 | 0.08 | 0.08 | 0.09 | 0.08 | 0.10 | 0.07 | 0.10 | 0.08 | 0.06 | 0.07 | 0.10 | 0.05 | 0.08 | 0.10 | 0.11 |
| 10 | 0.09 | 0.10 | 0.11 | 0.11 | 0.11 | 0.13 | 1.09 | 0.12 | 0.09 | 0.07 | 0.07 | 0.11 | 0.06 | 0.09 | 0.12 | 0.12 |
| 11 | 0.09 | 0.10 | 0.10 | 0.10 | 0.09 | 0.11 | 0.09 | 0.12 | 0.09 | 0.07 | 0.08 | 0.10 | 0.07 | 0.08 | 0.12 | 0.13 |
| 12 | 0.09 | 0.09 | 0.10 | 0.10 | 0.08 | 0.11 | 0.09 | 0.12 | 0.10 | 0.08 | 0.08 | 0.11 | 0.06 | 0.08 | 0.13 | 0.13 |
| 13 | 0.09 | 0.08 | 0.08 | 0.09 | 0.08 | 0.09 | 0.09 | 0.10 | 0.09 | 0.07 | 0.08 | 0.10 | 0.07 | 0.08 | 0.11 | 0.11 |
| 14 | 0.09 | 0.10 | 0.11 | 0.11 | 0.08 | 0.12 | 0.09 | 0.12 | 0.08 | 0.07 | 0.07 | 0.09 | 0.05 | 0.07 | 0.14 | 0.14 |
| 15 | 0.08 | 0.09 | 0.11 | 0.10 | 0.09 | 0.13 | 0.09 | 0.13 | 0.09 | 0.06 | 0.07 | 0.13 | 0.05 | 0.08 | 0.15 | 0.16 |
| 16 | 0.08 | 0.09 | 0.08 | 0.10 | 0.08 | 0.14 | 0.09 | 0.11 | 0.08 | 0.07 | 0.07 | 0.09 | 0.04 | 0.06 | 0.12 | 0.12 |
| 17 | 0.08 | 0.10 | 0.09 | 0.11 | 0.09 | 0.13 | 0.09 | 0.13 | 0.08 | 0.08 | 0.08 | 0.11 | 0.04 | 0.09 | 0.12 | 0.13 |
| 18 | 0.09 | 0.09 | 0.15 | 0.29 | 0.07 | 0.13 | 0.62 | 0.28 | 0.08 | 0.07 | 0.13 | 0.12 | 0.04 | 0.08 | 0.19 | 0.18 |
| 19 | 0.08 | 0.09 | 0.10 | 0.10 | 0.09 | 0.16 | 0.12 | 0.13 | 0.08 | 0.07 | 0.07 | 0.14 | 0.04 | 0.08 | 0.15 | 0.14 |
| 20 | 0.07 | 0.06 | 0.06 | 0.07 | 0.06 | 0.08 | 0.08 | 0.11 | 0.08 | 0.07 | 0.07 | 0.09 | 0.04 | 0.06 | 0.09 | 0.10 |
| 21 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 22 | 0.07 | 0.07 | 0.07 | 0.08 | 0.08 | 0.09 | 0.09 | 0.11 | 0.08 | 0.07 | 0.08 | 0.10 | 0.04 | 0.07 | 0.11 | 0.11 |
| 23 | 0.09 | 0.09 | 0.11 | 0.11 | 0.08 | 0.16 | 0.09 | 0.12 | 0.08 | 0.07 | 0.07 | 0.10 | 0.05 | 0.07 | 0.14 | 0.14 |
| 24 | 0.07 | 0.09 | 0.12 | 0.12 | 0.08 | 0.12 | 0.08 | 0.11 | 0.08 | 0.07 | 0.07 | 0.10 | 0.05 | 0.10 | 0.13 | 0.15 |
| 25 | 0.07 | 0.09 | 0.08 | 0.08 | 0.06 | 0.11 | 0.07 | 0.12 | 0.07 | 0.06 | 0.06 | 0.06 | 0.06 | 0.07 | 0.11 | 0.12 |
| 26 | 0.07 | 0.09 | 0.07 | 0.08 | 0.08 | 0.10 | 0.07 | 0.10 | 0.07 | 0.07 | 0.06 | 0.05 | 0.07 | 0.08 | 0.09 | 0.11 |
| 27 | 0.07 | 0.08 | 0.07 | 0.08 | 0.06 | 0.11 | 0.07 | 0.08 | 0.06 | 0.06 | 0.06 | 0.06 | 0.08 | 0.06 | 0.09 | 0.10 |
| 28 | 0.07 | 0.08 | 0.08 | 0.08 | 0.07 | 0.50 | 0.07 | 0.10 | 0.07 | 0.06 | 0.06 | 0.06 | 0.07 | 0.06 | 0.09 | 0.10 |
| 29 | 0.42 | 0.36 | 0.45 | 0.53 | 0.22 | 0.95 | 0.29 | 0.14 | 0.21 | 0.11 | 0.09 | 0.10 | 0.09 | 0.25 | 0.59 | 0.62 |
| 30 | 0.07 | 0.07 | 0.06 | 0.07 | 0.06 | 0.09 | 0.07 | 0.09 | 0.07 | 0.07 | 0.07 | 0.07 | 0.08 | 0.08 | 0.09 | 0.12 |
| 31 | 0.05 | 0.09 | 0.09 | 0.08 | 0.06 | 0.09 | 0.07 | 0.08 | 0.06 | 0.06 | 0.06 | 0.06 | 0.06 | 0.06 | 0.09 | 0.09 |
| 32 | 0.06 | 0.08 | 0.06 | 0.25 | 0.06 | 0.09 | 0.06 | 0.07 | 0.06 | 0.05 | 0.06 | 0.05 | 0.07 | 0.06 | 0.09 | 0.09 |
| 33 | 0.07 | 0.08 | 0.06 | 0.07 | 0.06 | 0.08 | 0.06 | 0.07 | 0.06 | 0.05 | 0.06 | 0.05 | 0.07 | 0.06 | 0.08 | 0.09 |
| 34 | 0.07 | 0.07 | 0.05 | 0.06 | 0.06 | 0.06 | 0.06 | 0.07 | 0.06 | 0.09 | 0.06 | 0.15 | 0.06 | 0.06 | 0.08 | 0.08 |
| 35 | 0.18 | 0.17 | 0.27 | 1.40 | 0.25 | 0.64 | 2.10 | 0.64 | 0.10 | 0.10 | 1.22 | 1.11 | 0.05 | 0.47 | 0.95 | 0.86 |
| 36 | 0.07 | 0.07 | 0.06 | 0.07 | 0.06 | 0.11 | 0.07 | 0.08 | 0.09 | 0.06 | 0.06 | 0.07 | 0.07 | 0.07 | 0.08 | 0.08 |
| 37 | 0.10 | 0.11 | 0.11 | 0.12 | 0.09 | 0.14 | 0.12 | 0.14 | 0.09 | 0.08 | 0.08 | 0.20 | 0.10 | 0.08 | 0.14 | 0.14 |
| 38 | 0.07 | 0.08 | 0.08 | 0.09 | 0.08 | 0.11 | 0.09 | 0.11 | 0.08 | 0.07 | 0.07 | 0.07 | 0.09 | 0.08 | 0.19 | 0.11 |
| 39 | 0.08 | 0.09 | 0.07 | 0.09 | 0.07 | 0.37 | 0.09 | 0.10 | 0.08 | 0.09 | 0.09 | 0.08 | 0.09 | 0.09 | 0.11 | 0.13 |
| 40 | 0.09 | 0.16 | 0.24 | 0.12 | 0.11 | 0.16 | 0.09 | 0.12 | 0.09 | 0.09 | 0.08 | 0.08 | 0.10 | 0.09 | 0.15 | 0.16 |
| 41 | 0.08 | 0.12 | 0.10 | 0.11 | 0.10 | 0.20 | 0.09 | 0.15 | 0.09 | 0.07 | 0.09 | 0.08 | 0.11 | 0.09 | 0.15 | 0.16 |
| 42 | 0.08 | 0.11 | 0.09 | 0.10 | 0.17 | 0.23 | 0.11 | 0.19 | 0.27 | 0.12 | 0.10 | 0.09 | 0.16 | 0.42 | 0.35 | 1.24 |
| 43 | 0.09 | 0.21 | 0.11 | 0.11 | 0.10 | 0.41 | 0.10 | 0.14 | 0.08 | 0.08 | 0.08 | 0.08 | 0.12 | 0.11 | 0.20 | 0.25 |
| 44 | 0.09 | 0.11 | 0.11 | 0.12 | 0.98 | 0.15 | 0.10 | 0.14 | 0.09 | 0.09 | 0.08 | 0.08 | 0.12 | 0.13 | 0.17 | 1.25 |
| 45 | 0.08 | 0.06 | 0.06 | 0.12 | 0.10 | 0.17 | 0.10 | 0.14 | 0.09 | 0.08 | 0.06 | 0.09 | 0.11 | 0.09 | 0.16 | 0.17 |
| 46 | 0.08 | 0.08 | 0.07 | 0.09 | 0.08 | 0.22 | 0.10 | 0.12 | 0.08 | 0.07 | 0.07 | 0.07 | 0.09 | 0.08 | 0.19 | 0.26 |
| 47 | 0.07 | 0.09 | 0.08 | 0.09 | 0.09 | 0.13 | 0.09 | 0.12 | 0.07 | 0.07 | 0.07 | 0.07 | 0.10 | 0.09 | 0.15 | 0.19 |
| 48 | 0.08 | 0.11 | 0.10 | 0.12 | 0.10 | 0.16 | 0.11 | 0.13 | 0.08 | 0.09 | 0.08 | 0.08 | 0.12 | 0.09 | 0.15 | 0.15 |
| 49 | 0.09 | 0.11 | 0.10 | 0.11 | 0.10 | 0.15 | 0.15 | 0.15 | 0.08 | 0.08 | 0.07 | 0.08 | 0.10 | 0.11 | 0.26 | 0.25 |
| 50 | 0.07 | 0.09 | 0.08 | 0.09 | 0.08 | 0.86 | 0.29 | 0.18 | 0.08 | 0.07 | 0.07 | 0.07 | 0.10 | 0.20 | 0.63 | 0.46 |
| 51 | 0.06 | 0.09 | 0.08 | 0.10 | 0.12 | 0.24 | 0.13 | 0.13 | 0.07 | 0.07 | 0.08 | 0.08 | 0.11 | 0.09 | 0.24 | 0.78 |
| 52 | 0.10 | 0.13 | 0.11 | 0.11 | 0.10 | 0.20 | 0.11 | 0.13 | 0.10 | 0.09 | 0.08 | 0.08 | 0.14 | 0.11 | 0.21 | 0.38 |
| 53 | 0.12 | 0.12 | 0.09 | 0.10 | 0.08 | 0.16 | 0.09 | 0.11 | 0.09 | 0.10 | 0.08 | 0.07 | 0.09 | 0.10 | 0.16 | 0.15 |
| 54 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 55 | 0.10 | 0.10 | 0.07 | 0.08 | 0.07 | 0.14 | 0.09 | 0.08 | 0.35 | 0.08 | 0.07 | 0.07 | 0.08 | 0.09 | 0.14 | 0.12 |
| 56 | 0.10 | 0.16 | 0.11 | 0.11 | 0.09 | 0.18 | 0.09 | 0.11 | 0.10 | 0.09 | 0.09 | 0.09 | 0.10 | 0.10 | 0.18 | 0.15 |
| 57 | 0.14 | 0.12 | 0.10 | 0.12 | 0.09 | 0.17 | 0.11 | 0.12 | 0.11 | 0.08 | 0.09 | 0.09 | 0.10 | 0.12 | 0.19 | 0.43 |
| 58 | 0.07 | 0.06 | 0.06 | 0.07 | 0.06 | 0.07 | 0.07 | 0.08 | 0.07 | 0.06 | 0.08 | 0.08 | 0.04 | 0.06 | 0.08 | 0.09 |
| 59 | 0.06 | 0.05 | 0.05 | 0.06 | 0.06 | 0.06 | 0.06 | 0.08 | 0.06 | 0.05 | 0.05 | 0.06 | 0.04 | 0.05 | 0.07 | 0.08 |
| 60 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 61 | 0.07 | 0.06 | 0.06 | 0.08 | 0.06 | 0.10 | 0.07 | 0.10 | 0.08 | 0.05 | 0.06 | 0.08 | 0.04 | 0.07 | 0.12 | 0.12 |
| 62 | 0.07 | 0.07 | 0.06 | 0.09 | 0.07 | 0.08 | 0.07 | 0.09 | 0.08 | 0.05 | 0.07 | 0.11 | 0.05 | 0.07 | 0.11 | 0.14 |
| 63 | 0.11 | 0.16 | 0.12 | 0.13 | 0.11 | 0.16 | 0.11 | 0.05 | 0.08 | 0.08 | 0.08 | 0.08 | 0.11 | 0.09 | 0.15 | 0.15 |
| 64 | 0.09 | 0.08 | 0.08 | 0.09 | 0.07 | 0.10 | 0.08 | 0.09 | 0.08 | 0.08 | 0.08 | 0.07 | 0.08 | 0.08 | 0.11 | 0.12 |
| 65 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 66 | 0.10 | 0.12 | 0.10 | 0.10 | 0.10 | 0.14 | 0.12 | 0.16 | 0.10 | 0.09 | 0.09 | 0.09 | 0.11 | 0.10 | 0.13 | 0.15 |
| 67 | 0.10 | 0.11 | 0.09 | 0.12 | 0.10 | 0.15 | 0.12 | 0.15 | 0.10 | 0.09 | 0.09 | 0.09 | 0.10 | 0.10 | 0.21 | 0.24 |
| 68 | 0.11 | 0.14 | 0.16 | 0.16 | 0.13 | 0.23 | 0.13 | 0.19 | 0.10 | 0.10 | 0.09 | 0.10 | 0.12 | 0.12 | 0.20 | 0.21 |
| 69 | 0.10 | 0.15 | 0.16 | 0.14 | 0.12 | 0.51 | 0.11 | 0.13 | 0.09 | 0.08 | 0.08 | 0.09 | 0.10 | 0.11 | 0.15 | 0.21 |
| 70 | 0.09 | 0.11 | 0.11 | 0.11 | 0.09 | 0.19 | 0.12 | 0.12 | 0.08 | 0.08 | 0.08 | 0.09 | 0.10 | 0.11 | 0.18 | 0.20 |
| 71 | 0.10 | 0.13 | 0.14 | 0.14 | 0.11 | 0.20 | 0.11 | 0.17 | 0.10 | 0.08 | 0.10 | 0.09 | 0.13 | 0.14 | 0.21 | 0.20 |
| 72 | 0.10 | 0.15 | 0.12 | 0.13 | 0.10 | 0.19 | 0.12 | 0.16 | 0.08 | 0.08 | 0.09 | 0.09 | 0.11 | 0.11 | 0.18 | 0.19 |
| 73 | 0.08 | 0.09 | 0.09 | 0.10 | 0.09 | 0.12 | 0.10 | 0.13 | 0.09 | 0.08 | 0.07 | 0.08 | 0.11 | 0.09 | 0.13 | 0.14 |
| 74 | 0.08 | 0.09 | 0.07 | 0.08 | 0.08 | 0.10 | 0.09 | 0.11 | 0.09 | 0.08 | 0.07 | 0.08 | 0.08 | 0.08 | 0.09 | 0.11 |
| 75 | 0.11 | 0.12 | 0.12 | 0.11 | 0.10 | 0.17 | 0.44 | 0.15 | 0.10 | 0.10 | 0.10 | 0.09 | 0.11 | 0.10 | 0.15 | 0.16 |
| 76 | 0.10 | 0.13 | 0.15 | 0.14 | 0.11 | 0.28 | 0.13 | 0.14 | 0.11 | 0.09 | 0.08 | 0.09 | 0.12 | 0.26 | 0.01 | 0.22 |
| 77 | 0.14 | 0.14 | 0.15 | 0.12 | 0.09 | 0.18 | 0.12 | 0.13 | 0.18 | 0.07 | 0.07 | 0.08 | 0.08 | 0.09 | 0.11 | 0.14 |

*Values are given as the optical density (OD) at 405 nm. OD values over 0.500 are considered positive and have been written in bold.

TABLE 3
Analysis of human antibody reactivities in EIA to overlapping synthetic peptides corresponding to the complete open
reading frame 2 (ORF2) of the TT virus*

| TTV | | | | | | | | | | | | | | | | |
|---|---|---|---|---|---|---|---|---|---|---|---|---|---|---|---|---|
| ORF1 | Samples negative for TTV DNA by PCR | | | | | | | | Samples positive for TTV DNA by PCR | | | | | | | |
| peptide | 1 | 2 | 3 | 4 | 5 | 6 | 7 | 11 | 9 | 18 | 36 | 48 | 98 | 110 | 155 | 157 |
| 1 | 0.09 | 0.10 | 0.13 | 0.11 | 0.10 | 0.16 | 0.09 | 0.12 | 0.08 | 0.08 | 0.09 | 0.08 | 0.08 | 0.08 | 0.14 | 0.14 |
| 2 | 0.10 | 0.11 | 0.11 | 0.10 | 0.08 | 0.12 | 0.09 | 0.14 | 0.08 | 0.08 | 0.08 | 0.08 | 0.08 | 0.09 | 0.14 | 0.13 |
| 3 | 0.08 | 0.10 | 0.11 | 0.10 | 0.08 | 0.29 | 0.28 | 0.14 | 0.10 | 0.08 | 0.13 | 0.08 | 0.11 | 0.10 | 0.14 | 0.04 |
| 4 | 0.09 | 0.09 | 0.08 | 0.08 | 0.07 | 0.09 | 0.07 | 0.09 | 0.08 | 0.07 | 0.07 | 0.07 | 0.07 | 0.07 | 0.10 | 0.10 |
| 5 | 0.08 | 0.08 | 0.08 | 0.08 | 0.07 | 0.10 | 0.08 | 0.10 | 0.07 | 0.07 | 0.07 | 0.07 | 0.07 | 0.08 | 0.12 | 0.13 |
| 6 | 0.07 | 0.10 | 0.08 | 0.10 | 0.07 | 0.10 | 0.07 | 0.10 | 0.08 | 0.28 | 0.09 | 0.08 | 0.10 | 0.09 | 0.09 | 0.11 |
| 7 | 0.11 | 0.10 | 0.11 | 0.14 | 0.91 | 0.13 | 0.10 | 0.11 | 0.08 | 0.09 | 0.10 | 0.11 | 0.10 | 0.09 | 0.10 | 0.13 |
| 8 | 0.09 | 0.10 | 0.11 | 0.13 | 0.10 | 0.12 | 0.10 | 0.14 | 0.09 | 0.08 | 0.10 | 0.11 | 0.10 | 0.10 | 0.11 | 0.12 |
| 9 | 0.08 | 0.09 | 0.09 | 0.09 | 0.09 | 0.10 | 0.09 | 0.11 | 0.09 | 0.08 | 0.08 | 0.09 | 0.09 | 0.09 | 0.13 | 0.14 |
| 10 | 0.09 | 0.11 | 0.10 | 0.09 | 0.08 | 0.11 | 0.08 | 0.10 | 0.09 | 0.09 | 0.07 | 0.08 | 0.08 | 0.08 | 0.11 | 0.13 |
| 11 | 0.10 | 0.10 | 0.11 | 0.08 | 0.09 | 0.09 | 0.09 | 0.11 | 0.10 | 0.12 | 0.09 | 0.07 | 0.09 | 0.09 | 0.14 | 0.14 |
| 12 | 0.12 | 0.09 | 0.09 | 0.08 | 0.09 | 0.09 | 0.08 | 0.07 | 0.13 | 0.09 | 0.08 | 0.10 | 0.14 | 0.12 | 0.16 | 0.14 |
| 13 | 0.07 | 0.09 | 0.09 | 0.08 | 0.08 | 0.10 | 0.09 | 0.11 | 0.07 | 0.08 | 0.07 | 0.08 | 0.09 | 0.08 | 0.12 | 0.12 |
| 14 | 0.08 | 0.07 | 0.08 | 0.07 | 0.07 | 0.08 | 0.08 | 0.10 | 0.07 | 0.07 | 0.07 | 0.07 | 0.07 | 0.07 | 0.10 | 0.10 |
| 15 | 0.08 | 0.08 | 0.08 | 0.07 | 0.07 | 0.09 | 0.07 | 0.10 | 0.08 | 0.07 | 0.07 | 0.07 | 0.08 | 0.08 | 0.10 | 0.12 |
| 16 | 0.08 | 0.09 | 0.09 | 0.07 | 0.07 | 0.09 | 0.08 | 0.09 | 0.08 | 0.08 | 0.08 | 0.07 | 0.08 | 0.08 | 0.12 | 0.11 |
| 17 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 18 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 | 0.00 |
| 19 | 0.09 | 0.12 | 0.17 | 0.12 | 0.08 | 0.51 | 0.57 | 0.15 | 0.08 | 0.07 | 0.33 | 0.07 | 0.08 | 0.08 | 0.14 | 0.14 |
| 20 | 0.09 | 0.10 | 0.09 | 0.08 | 0.07 | 0.10 | 0.07 | 0.09 | 0.10 | 0.10 | 0.08 | 0.08 | 0.08 | 0.10 | 0.14 | 0.16 |

*Values have been given as the optical density (OD) at 405 nm. OD values over 0.500 are considered positive and have been written in bold.

TABLE 4
Sequences of TTV peptides that are reactive with
TTV infected human serum

| Peptide no. | Peptide sequence | SEQ ID NO: |
|---|---|---|
| ORF1 | | |
| 10 | VLICGENTVSRNYATHS | SEQ ID NO:11 |
| 18 | KINTMPPFLDTELTAPS | SEQ ID NO:12 |
| 29 | PDEKSQREILLNKIASY | SEQ ID NO:13 |
| 35 | TATTTTYAYPGTNRPPV | SEQ ID NO:1 |
| 42 | GLYSSIWLSPGRSYFET | SEQ ID NO:14 |
| 44 | YTDIKYNPFTDRGEGNM | SEQ ID NO:15 |
| 50 | DQNIHMNARLLIRSPFT | SEQ ID NO:16 |
| 51 | LIRSPFTDPQLLVHTDP | SEQ ID NO:17 |
| 69 | QKESLLFPPVKLLRRVP | SEQ ID NO:18 |
| ORF2 | | |
| 19 | EDGGAGGDADHGGAAGGP | SEQ ID NO:19 |

The peptide that was found to be reactive to most of the TTV infected serum samples was peptide 35, which has the sequence TATTTTYAYPGTNRPPV (SEQ ID NO: 1). The reactivity to peptide 35 was dependent on the dilution of the serum samples, however. (See TABLE 5). In another set of experiments, it was determined that the reactivity of the human serum samples to the peptide on the microplate was inhibited by the addition of the same peptide in solution, but not by a control peptide having a non-corresponding sequence. These data verify that the reactivity of the sequence TATTTTYAYPGTNRPPV (SEQ ID NO: 1) with anti-TTV antibodies present in human serum from TTV infected individuals is specific.

TABLE 5
Analysis of the reactivities of serial dilutions of three human serum
samples with the TTV peptide TATTTTYAYPGTNRPPV
(SEQ ID NO: 1)*

| Dilution of | | | | | | |
|---|---|---|---|---|---|---|
| serum | Human serum sample | | | | | |
| sample | P4 | SD | P6 | SD | P7 | SD |
| 1:100 | 1.285 | 0.072 | 0.687 | 0.082 | 1.782 | 0.054 |
| 1:200 | 0.758 | 0.056 | 0.375 | 0.003 | 1.23 | 0.02 |
| 1:400 | 0.411 | 0.021 | 0.19 | 0.007 | 0.79 | 0.018 |
| 1:800 | 0.234 | 0.008 | 0.104 | 0.003 | 0.45 | 0.002 |
| 1:1600 | 0.131 | 0.005 | 0.067 | 0.001 | 0.246 | 0.013 |
| 1:3200 | 0.076 | 0.003 | 0.049 | | 0.131 | 0.006 |
| 1:6400 | 0.058 | 0.001 | 0.043 | | 0.087 | |
| 1:12800 | 0.046 | | 0.041 | | 0.061 | |

*Values are given as the OD and standard deviation (SD) at 405 nm.

The reactivity to the TATTTTYAYPGTNRPPV (SEQ ID NO: 1) peptide was further characterized by mapping the reactivity of deletion and substitution peptide analogues. Deletion analysis showed that the reactive region of SEQ ID NO: 1 contained the sequence YAYPGTNRPPV (SEQ ID NO: 2) (See TABLE 6). Using alanine substitution analogues, the Pro-Val sequence was found to be important for the binding of human antibodies (See TABLE 6).

TABLE 6
Analysis of the reactivities of three human serum
samples with the deletion and alanine substitution
analogues of the TTV peptide TATTTTYAYPGTNRPPV
(SEQ ID NO:1)*

| SEQ | Deletion or substitution | Human serum sample | | |
|---|---|---|---|---|
| ID NO: | peptide analogue | P4 | P7 | P36 |
| 1 | TATTTTYAYPGTNRPPV | 0.839 | 1.845 | 0.825 |
| 20 | TATTTTYAYPGTNRPP | 0.096 | 0.144 | 0.086 |
| 21 | TATTTTYAYPGTNRP | 0.100 | 0.099 | 0.078 |
| 22 | TATTTTYAYPGTNR | 0.092 | 0.103 | 0.078 |
| 23 | TATTTTYAYPGTN | 0.186 | 0.888 | 0.083 |
| 24 | TATTTTYAYPGT | 0.095 | 0.087 | 0.072 |
| 25 | TATTTTYAYPG | 0.095 | 0.085 | 0.074 |
| 26 | TATTTTYAYP | 0.095 | 0.096 | 0.082 |
| 27 | TATTTTYAY | 0.098 | 0.089 | 0.083 |
| 28 | TATTTTYA | 0.115 | 0.089 | 0.090 |
| 29 | TATTTTY | 0.142 | 0.105 | 0.076 |
| 3 | TATTTT | 0.108 | 0.093 | 0.082 |
| 30 | TATTT | 0.101 | 0.091 | 0.078 |
| 31 | TATT | 0.099 | 0.105 | 0.076 |
| 32 | ATTTTYAYPGTNRPPV | 1.042 | 1.960 | 0.923 |
| 33 | TTTTYAYPGTNRPPV | 0.805 | 1.587 | 0.776 |
| 34 | TTTYAYPGTNRPPV | 0.697 | 1.488 | 0.810 |
| 35 | TTYAYPGTNRPPV | 0.748 | 1.659 | 0.722 |
| 36 | TYAYPGTNRPPV | 0.707 | 1.508 | 0.712 |
| 2 | YAYPGTNRPPV | 0.647 | 1.546 | 0.677 |
| 37 | AYPGTNRPPV | 0.662 | 1.488 | 0.669 |
| 38 | YPGTNRPPV | 0.300 | 1.091 | 0.406 |
| 39 | PGTNRPPV | 0.166 | 0.430 | 0.123 |
| 40 | GTNRPPV | 0.300 | 0.887 | 0.210 |
| 41 | TNRPPV | 0.110 | 0.146 | 0.056 |
| 42 | NRPPV | 0.135 | 0.242 | 0.076 |
| 43 | AATTTTYAYPGTNRPPV | 1.045 | 1.852 | 0.915 |
| 44 | TGTTTTYAYPGTNRPPV | 0.855 | 1.829 | 0.806 |
| 45 | TAATTTYAYPGTNRPPV | 0.897 | 1.675 | 0.764 |
| 46 | TATATTYAYPGTNRPPV | 0.971 | 1.722 | 0.824 |
| 47 | TATTATYAYPGTNRPPV | 1.076 | 1.867 | 0.955 |
| 48 | TATTTAYAYPGTNRPPV | 1.011 | 1.833 | 1.027 |
| 49 | TATTTTAAYPGTNRPPV | 0.898 | 1.619 | 0.901 |
| 50 | TATTTTYGYPGTNRPPV | 0.836 | 1.769 | 0.850 |
| 51 | TATTTTYAAPGTNRPPV | 0.899 | 1.697 | 0.903 |
| 52 | TATTTTYAYAGTNRPPV | 0.886 | 1.738 | 0.903 |
| 53 | TATTTTYAYPATNRPPV | 0.895 | 1.503 | 0.734 |
| 54 | TATTTTYAYPGANRPPV | 0.891 | 1.594 | 0.714 |
| 55 | TATTTTYAYPGTARPPV | 1.226 | 1.723 | 0.696 |
| 56 | TATTTTYAYPGTNAPPV | 0.761 | 1.558 | 0.708 |
| 57 | TATTTTYAYPGTNRAPV | 0.720 | 1.551 | 0.812 |
| 58 | TATTTTYAYPGTNRPAV | 0.090 | 0.092 | 0.100 |
| 59 | TATTTTYAYPGTNRPPA | 0.108 | 0.105 | 0.095 |

*Values are given as the OD at 405 nm. Positive reactivities, i.e., more than 50% of the reactivity of the original peptide, have been written in bold.

To determine that the reactive peptides could be used to generate antibodies in mammals, immunization experiments were conducted in mice, as described in the following section.

Immunization and induction of TTV-specific antibodies in mammals

A total of five Balb/c mice were immunized intra-peritoneally with 100 μg of TTV peptide 35 (SEQ ID NO: 1) emulsified 1:1 in complete Freund's adjuvant. Venous blood samples were obtained on weeks 2, 4, and 6. A booster dose of 100 μg in incomplete Freund's adjuvant was given on week four. Out of the five mice tested, one developed detectable antibodies specific for TTV peptide 35 (SEQ ID NO: 1). The anti-peptide titers were 1:60 at week 4 and 1:2160 at week 6. Thus, TTV peptide 35 (SEQ ID NO: 1) was found to induce TTV peptide-specific antibodies in mammals. The following section describes more on the production of antibodies specific for TTV.

Production of antibodies specific for TTV

For the production of antibodies, various hosts including goats, rabbits, rats, mice, etc. can be immunized by injection with any of the peptides described herein. Depending on the host species, various adjuvants can be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. BCG (Bacillus Calmette-Guerin) and Corynebacterium parvum are also potentially useful adjuvants.

Monoclonal antibodies directed to the peptides described herein can be prepared using any technique that provides for the production of antibody molecules by continuous cell lines in culture These include, but are not limited to, the hybridoma technique originally described by Koehler and Milstein (Nature 256:495-497 (1975), the human B-cell hybridoma technique (Kosbor et al. Immunol Today 4:72 (1983); Cote et al Proc Natl Acad Sci 80:2026-2030 (1983), and the EBV-hybridoma technique Cole et al. Monoclonal Antibodies and Cancer Therapy, Alan R. Liss Inc, New York N.Y., pp 77-96 (1985), all of which are hereby expressly incorporated by reference in their entireties.

In addition, techniques developed for the production of “chimeric antibodies”, the splicing of mouse antibody genes to human antibody genes to obtain a molecule with appropriate antigen specificity and biological activity can be used. (Morrison et al. Proc Natl Acad Sci 81:6851-6855 (1984); Neuberger et al. Nature 312:604-608(1984); Takeda et al. Nature 314:452-454(1985), all of which are hereby expressly incorporated by reference in their entireties. Alternatively, techniques described for the production of single chain antibodies (U.S. Pat. No. 4,946,778), herein expressly incorporated by reference in its entirety, can be adapted to produce peptide-specific single chain antibodies. Antibodies can also be produced by inducing in vivo production in the lymphocyte population or by screening recombinant immunoglobulin libraries or panels of highly specific binding reagents as disclosed in Orlandi et al., Proc Natl Acad Sci 86: 3833-3837 (1989), and Winter G. and Milstein C; Nature 349:293-299 (1991), all of which are hereby expressly incorporated by reference in their entireties.

Antibody fragments that contain specific binding sites for the peptides described herein can also be generated. For example, such fragments include, but are not limited to, the F(ab′)₂ fragments that can be produced by pepsin digestion of the antibody molecule and the Fab fragments that can be generated by reducing the disulfide bridges of the F(ab′)₂ fragments. Alternatively, Fab expression libraries can be constructed to allow rapid and easy identification of monoclonal Fab fragments with the desired specificity. (Huse W. D. et al. Science 256:1275-1281 (1989), hereby expressly incorporated by reference in its entirety).

By one approach, monoclonal antibodies to an isolated or purified peptide of SEQ ID NO: 1 are made as follows. Briefly, a mouse is repetitively inoculated with a few micrograms of the peptide over a period of a few weeks. The mouse is then sacrificed, and the antibody producing cells of the spleen isolated. The spleen cells are fused in the presence of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, E., Meth. Enzymol. 70:419 (1980), hereby expressly incorporated by reference in its entirety. Selected positive clones can be expanded and their monoclonal antibody product harvested for use. Detailed procedures for monoclonal antibody production are described in Davis, L. et al. Basic Methods in Molecular Biology Elsevier, N.Y. Section 21-2, hereby expressly incorporated by reference in its entirety.

Polyclonal antiserum containing antibodies to heterogenous epitopes of a single protein can be prepared by immunizing suitable animals with the isolated or purified peptide of SEQ ID NO: 1. Effective polyclonal antibody production is affected by many factors related both to the antigen and the host species. For example, small molecules tend to be less immunogenic than others and can require the use of carriers and adjuvant. Also, host animals vary in response to site of inoculations and dose, with both inadequate or excessive doses of antigen resulting in low titer antisera. Small doses (ng level) of antigen administered at multiple intradermal sites appears to be most reliable. An effective immunization protocol for rabbits can be found in Vaitukaitis, J. et al. J Clin. Endocrinol. Metab. 33:988-991 (1971), hereby expressly incorporated by reference in its entirety.

Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973), hereby expressly incorporated by reference in its entirety. Plateau concentration of antibody is usually in the range of 0.1 to 0.2 mg/ml of serum (about 12 μM). Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980), hereby expressly incorporated by reference in its entirety. The following section describes more about the manufacture of medicaments containing the peptides or antibodies described herein.

The manufacture and dose of therapeutic and prophylactic agents

The peptides and antibodies described herein are suitable for treatment of subjects either as a preventive measure to avoid TTV, or as a therapeutic to treat subjects already afflicted with TTV. These pharmacologically active compounds can be processed in accordance with conventional methods of galenic pharmacy to produce medicinal agents for administration to subjects, e.g., mammals including humans. The active ingredients can be incorporated into a pharmaceutical product with and without modification. Further, the manufacture of pharmaceuticals or therapeutic agents that deliver the pharmacologically active compounds described herein by several routes are aspects of the invention. For example, and not by way of limitation, DNA, RNA, and viral vectors having sequences encoding the peptides or antibodies described herein are embodiments. Nucleic acids encoding the peptides or antibodies described herein can be administered alone or in combination with other active ingredients.

The compounds of this invention can be employed in admixture with conventional excipients, i.e., pharmaceutically acceptable organic or inorganic carrier substances suitable for parenteral, enteral (e.g., oral) or topical application that do not deleteriously react with the pharmacologically active ingredients of this invention. Suitable pharmaceutically acceptable carriers include, but are not limited to, water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Many more suitable vehicles are described in Remmington's Pharmaceutical Sciences, 15th Edition, Easton:Mack Publishing Company, pages 1405-1412 and 1461-1487(1975) and The National Formulary XIV, 14th Edition, Washington, American Pharmaceutical Association (1975), herein incorporated by reference. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The effective dose and method of administration of a particular pharmaceutical formulation can vary based on the individual patient and the type and stage of the disease, as well as other factors known to those of skill in the art. Therapeutic efficacy and toxicity of such compounds can be determined by standard pharmaceutical procedures in cell cultures or experimental animals, e.g., ED50 (the dose therapeutically effective in 50% of the population). The dosage of such compounds lies preferably within a range of circulating concentrations that include the ED50 with no toxicity. The dosage varies within this range depending upon type of peptide or antibody, the dosage form employed, sensitivity of the patient, and the route of administration.

Normal dosage amounts may vary from approximately 1 to 100,000 micrograms, up to a total dose of about 10 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5, 6 g, 7 g, 8 g, 9 g, and 10 g. Additionally, the concentrations of the peptides or antibodies described herein can be quite high in embodiments that administer the agents in a topical form. Molar concentrations of the peptides or antibodies described herein can be used with some embodiments. Desirable concentrations for topical administration and/or for coating medical equipment range from 100 μM to 800 mM. Preferable concentrations for these embodiments range from 500 μM to 500 mM. For example, preferred concentrations for use in topical applications and/or for coating medical equipment include 500 μM, 550 μM, 600 μM, 650 μM, 700 μM, 750 μM, 800 μM, 850 μM, 900 μM, 1 mM, 5 mM, 10 mM, 15 mM, 20 mM, 25 mM, 30 mM, 35 mM, 40 mM, 45 mM, 50 mM, 60 mM, 70 mM, 80 mM, 90 mM, 100 mM, 120 mM, 130 mM, 140 mM, 150 mM, 160 mM, 170 mM, 180 mM, 190 mM, 200 mM, 300 mM, 325 mM, 350 mM, 375 mM, 400 mM, 425 mM, 450 mM, 475 mM, and 500 mM.

In some embodiments, the dose of the peptides or antibodies described herein preferably produces a tissue or blood concentration or both from approximately 0.1 μM to 500 mM. Desirable doses produce a tissue or blood concentration or both of about 1 to 800 μM. Preferable doses produce a tissue or blood concentration of greater than about 10 μM to about 500 μM. Preferable doses are, for example, the amount of the peptide or antibody to achieve a tissue or blood concentration or both of 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 120 μM, 130 μM, 140 μM, 145 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration of greater than 800 μM are not preferred, they can be used with some embodiments of the invention. A constant infusion of the peptides or antibodies described herein can also be provided so as to maintain a stable concentration in the tissues as measured by blood levels.

The exact dosage is chosen by the individual physician in view of the patient to be treated. Dosage and administration are adjusted to provide sufficient levels of the active moiety or to maintain the desired effect. Additional factors that can be taken into account include the severity of the disease state of the patient, age, and weight of the patient; diet, time and frequency of administration, drug combination(s), reaction sensitivities, and tolerance/response to therapy. Short acting pharmaceutical compositions are administered daily whereas long acting pharmaceutical compositions are administered every 2, 3 to 4 days, every week, or once every two weeks. Depending on half-life and clearance rate of the particular formulation, the pharmaceutical compositions of the invention are administered once, twice, three, four, five, six, seven, eight, nine, ten or more times per day.

Routes of administration of the pharmaceuticals of the invention include, but are not limited to, transdermal, parenteral, gastrointestinal, transbronchial, and transalveolar. Transdermal administration is accomplished by application of a cream, rinse, gel, etc. capable of allowing the pharmacologically active compounds to penetrate the skin. Parenteral routes of administration include, but are not limited to, electrical or direct injection such as direct injection into a central venous line, intravenous, intramuscular, intraperitoneal, intradermal, or subcutaneous injection. Gastrointestinal routes of administration include, but are not limited to, ingestion and rectal. Transbronchial and transalveolar routes of administration include, but are not limited to, inhalation, either via the mouth or intranasally.

Compositions having the pharmacologically active compounds of this invention that are suitable for transdermal administration include, but are not limited to, pharmaceutically acceptable suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference. Examples of suitable transdermal devices are described, for instance, in U.S. Pat. No. 4,818,540 issued Apr. 4, 1989 to Chinen, et al., herein incorporated by reference.

Compositions having the pharmacologically active compounds of this invention that are suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, intradermal, or subcutaneous injection.

Compositions having the pharmacologically active compounds of this invention that are suitable for transbronchial and transalveolar administration include, but not limited to, various types of aerosols for inhalation. Devices suitable for transbronchial and transalveolar administration of these are also embodiments. Such devices include, but are not limited to, atomizers and vaporizers. Many forms of currently available atomizers and vaporizers can be readily adapted to deliver compositions having the pharmacologically active compounds of the invention.

Compositions having the pharmacologically active compounds of this invention that are suitable for gastrointestinal administration include, but not limited to, pharmaceutically acceptable powders, pills or liquids for ingestion and suppositories for rectal administration. Due to the ease of use, gastrointestinal administration, particularly oral, is a preferred embodiment. Once the pharmaceutical comprising the peptides or antibodies described herein has been obtained, it can be administered to a subject in need to treat or prevent TTV infection.

Aspects of the invention also include a coating for medical equipment such as prosthetics, implants, and instruments. Coatings suitable for use in medical devices can be provided by a gel or powder containing the peptides or antibodies described herein or by polymeric coating into which the peptides or antibodies described herein are suspended. Suitable polymeric materials for coatings or devices are those that are physiologically acceptable and through which a therapeutically effective amount of the peptides or antibodies can diffuse. Suitable polymers include, but are not limited to, polyurethane, polymethacrylate, polyamide, polyester, polyethylene, polypropylene, polystyrene, polytetrafluoroethylene, polyvinyl-chloride, cellulose acetate, silicone elastomers, collagen, silk, etc. Such coatings are described, for instance, in U.S. Pat. No. 4,612,337, issued Sep. 16, 1986 to Fox et al. that is incorporated herein by reference in its entirety.

Although the invention has been described with reference to embodiments and examples, it should be understood that various modifications can be made without departing from the spirit of the invention. Accordingly, the invention is limited only by the following claims. All references cited herein are hereby expressly incorporated by reference.

What is claimed is:

1. An isolated or purified peptide consisting of an amino acid sequence selected from the group consisting of: SEQ ID NO: 1, SEQ ID NO:2, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 60, SEQ ID NO: 61, SEQ ID NO: 62, SEQ ID NO: 63, and SEQ ID NO: 64.

2. The isolated or purified peptide of claim 1, wherein said amino acid sequence consists of SEQ. ID NO: 1.

3. The isolated or purified peptide of claim 1, wherein said amino acid sequence consists of SEQ. ID NO: 2.

4. The isolated or purified peptide of claim 1, wherein said amino acid sequence consists of SEQ. ID NO: 11.

5. The isolated or purified peptide of claim 1, wherein said amino acid sequence consists of SEQ. ID NO: 12.

6. The isolated or purified peptide of claim 1, wherein said amino acid sequence consists of SEQ. ID NO: 13.

7. The isolated or purified peptide of claim 1, wherein said amino acid sequence consists of SEQ. ID NO: 14.

8. The isolated or purified peptide of claim 1, wherein said amino acid sequence consists of SEQ. ID NO: 15.

9. The isolated or purified peptide of claim 1, wherein said amino acid sequence consists of SEQ. ID NO: 16.

10. The isolated or purified peptide of claim 1, wherein said amino acid sequence consists of SEQ. ID NO: 17.

11. The isolated or purified peptide of claim 1, wherein said amino acid sequence consists of SEQ. ID NO: 18.

12. The isolated or purified peptide of claim 1, wherein said amino acid sequence consists of SEQ. ID NO: 19.

13. The isolated or purified peptide of claim 1, wherein said amino acid sequence consists of SEQ. ID NO: 60.

14. The isolated or purified peptide of claim 1, wherein said amino acid sequence consists of SEQ. ID NO: 61.

15. The isolated or purified peptide of claim 1, wherein said amino acid sequence consists of SEQ. ID NO: 62.

16. The isolated or purified peptide of claim 1, wherein said amino acid sequence consists of SEQ. ID NO: 63.

17. The isolated or purified peptide of claim 1, wherein said amino acid sequence consists of SEQ. ID NO: 64.

18. An isolated or purified antibody specific for a peptide consisting of the sequence of SEQ ID NO 1.

19. The antibody of claim 18, wherein said antibody is polyclonal.

20. The antibody of claim 18, wherein said antibody is monoclonal.

21. A method of making antibodies in a mammal comprising:
providing a peptide consisting of the sequence of SEQ ID NO: 1 to said mammal; and
identifying the presence of antibodies specific for said peptide.

22. The method of claim 21, wherein said mammal is a mouse.


SEQUENCE LISTING (64 sequences)

SEQ ID NO: 1 - protein, 17 aa, Artificial Sequence
  Peptide Immunogen
TATTTTYAYPGTNRPPV

SEQ ID NO: 2 - protein, 11 aa, Artificial Sequence
  Peptide Immunogen
YAYPGTNRPPV

SEQ ID NO: 3 - protein, 6 aa, Artificial Sequence
  Peptide Immunogen
TATTTT

SEQ ID NO: 4 - DNA, 24 bp, Artificial Sequence
  Amplification Primer
acagacagaggagaaggcaacatg

SEQ ID NO: 5 - DNA, 25 bp, Artificial Sequence
  Amplification Primer
ctggcattttaccatttccaaagtt

SEQ ID NO: 6 - DNA, 23 bp, Artificial Sequence
  Amplification Primer
taccayttagctctcattctwat

SEQ ID NO: 7 - DNA, 24 bp, Artificial Sequence
  Amplification Primer
ggcaacatgytrtggatagactgg

SEQ ID NO: 8 - DNA, 20 bp, Artificial Sequence
  Amplification Primer
acaggagachmaaacatasa

SEQ ID NO: 9 - protein, 770 aa, Artificial Sequence
  Peptide Immunogen
MAYGWWRRRRRRWRRWRRRPWRRRWRTRRRRPARRRGRRRNVRRRRRGGRWRRRYRRWKRKGRRRKKAKIIIRQWQPNYRRRCNIVGYIPVLICGENTVSRNYATHSDDTNYPGPFGGGMTTDKFTLRILYDEYKRFMNYWTASNEDLDLCRYLGVNLYFFRHPDVDFIIKINTMPPFLDTELTAPSIHPGMLALDKRARWIPSLKSRPGKKHYIKIRVGAPRMFTDKWYPQTDLCDMVLLTVYATAADMQYPFGSPLTDSVVVNFQVLQSMYDKTISILPDEKSQREILLNKIASYIPFYNTTQTIAQLKPFIDAGNVTSGATATTWASYINTTKFTTATTTTYAYPGTNRPPVTMLTCNDSWYRGTVYNTQIQQLPIKAAKLYLEATKTLLGNTFTNEDYTLEYHGGLYSSIWLSPGRSYFETTGAYTDIKYNPFTDRGEGNMLWIDWLSKKNMNYDKVQSKCLISDLPLWAAAYGYVEFCAKSTGDQNIHMNARLLIRSPFTDPQLLVHTDPTKGFVPYSLNFGNGKMPGGSSNVPIRMRAKWYPTLFHQQEVLEALAQSGPFAYHSDIKKVSLGMKYRFKWIWGGNPVRQQVVRNPCKETHSSGNRVPRSLQIVDPKYNSPELTFHTWDFRRGLFGPKAIQRMQQQPTTTDIFSAGRKRPRRDTEVYHSSQEGEQKESLLFPPVKLLRRVPPWEDSQQEESGSQSSEEETQTVSQQLKQQLQQQRILGVKLRLLFNQVQKIQQNQDINPTLLPRGGDLASLFQIAP

SEQ ID NO: 10 - protein, 201 aa, Artificial Sequence
  Peptide Immunogen
MAEFSTPVRSGEATEGDLRVPRAGAEGEFTHRSQGAIRARDWPGYGQGSEKSMFIGRHYRKKRALSLCAVRTTKKACKLLIVMWTPPRNDQHYLNWQWYSSILSSHAAMCGCPDAVAHFNHLASVLRAPQNPPPPGPQRNLPLRRLPALPAAPEAPGDRAPWPMAGGAEGEDGGAGGDADHGGAAGGPEDADLLDAVAAAE

SEQ ID NO: 11 - protein, 17 aa, Artificial Sequence
  Peptide Immunogen
VLICGENTVSRNYATHS

SEQ ID NO: 12 - protein, 17 aa, Artificial Sequence
  Peptide Immunogen
KINTMPPFLDTELTAPS

SEQ ID NO: 13 - protein, 17 aa, Artificial Sequence
  Peptide Immunogen
PDEKSQREILLNKIASY

SEQ ID NO: 14 - protein, 17 aa, Artificial Sequence
  Peptide Immunogen
GLYSSIWLSPGRSYFET

SEQ ID NO: 15 - protein, 17 aa, Artificial Sequence
  Peptide Immunogen
YTDIKYNPFTDRGEGNM

SEQ ID NO: 16 - protein, 17 aa, Artificial Sequence
  Peptide Immunogen
DQNIHMNARLLIRSPFT

SEQ ID NO: 17 - protein, 17 aa, Artificial Sequence
  Peptide Immunogen
LIRSPFTDPQLLVHTDP

SEQ ID NO: 18 - protein, 17 aa, Artificial Sequence
  Peptide Immunogen
QKESLLFPPVKLLRRVP

SEQ ID NO: 19 - protein, 18 aa, Artificial Sequence
  Peptide Immunogen
EDGGAGGDADHGGAAGGP

SEQ ID NO: 20 - protein, 16 aa, Artificial Sequence
  Peptide Immunogen
TATTTTYAYPGTNRPP

SEQ ID NO: 21 - protein, 15 aa, Artificial Sequence
  Peptide Immunogen
TATTTTYAYPGTNRP

SEQ ID NO: 22 - protein, 14 aa, Artificial Sequence
  Peptide Immunogen
TATTTTYAYPGTNR

SEQ ID NO: 23 - protein, 13 aa, Artificial Sequence
  Peptide Immunogen
TATTTTYAYPGTN

SEQ ID NO: 24 - protein, 12 aa, Artificial Sequence
  Peptide Immunogen
TATTTTYAYPGT

SEQ ID NO: 25 - protein, 11 aa, Artificial Sequence
  Peptide Immunogen
TATTTTYAYPG

SEQ ID NO: 26 - protein, 10 aa, Artificial Sequence
  Peptide Immunogen
TATTTTYAYP

SEQ ID NO: 27 - protein, 9 aa, Artificial Sequence
  Peptide Immunogen
TATTTTYAY

SEQ ID NO: 28 - protein, 8 aa, Artificial Sequence
  Peptide Immunogen
TATTTTYA

SEQ ID NO: 29 - protein, 7 aa, Artificial Sequence
  Peptide Immunogen
TATTTTY

SEQ ID NO: 30 - protein, 5 aa, Artificial Sequence
  Peptide Immunogen
TATTT

SEQ ID NO: 31 - protein, 4 aa, Artificial Sequence
  Peptide Immunogen
TATT

SEQ ID NO: 32 - protein, 16 aa, Artificial Sequence
  Peptide Immunogen
ATTTTYAYPGTNRPPV

SEQ ID NO: 33 - protein, 15 aa, Artificial Sequence
  Peptide Immunogen
TTTTYAYPGTNRPPV

SEQ ID NO: 34 - protein, 14 aa, Artificial Sequence
  Peptide Immunogen
TTTYAYPGTNRPPV

SEQ ID NO: 35 - protein, 13 aa, Artificial Sequence
  Peptide Immunogen
TTYAYPGTNRPPV

SEQ ID NO: 36 - protein, 12 aa, Artificial Sequence
  Peptide Immunogen
TYAYPGTNRPPV

SEQ ID NO: 37 - protein, 10 aa, Artificial Sequence
  Peptide Immunogen
AYPGTNRPPV

SEQ ID NO: 38 - protein, 9 aa, Artificial Sequence
  Peptide Immunogen
YPGTNRPPV

SEQ ID NO: 39 - protein, 8 aa, Artificial Sequence
  Peptide Immunogen
PGTNRPPV

SEQ ID NO: 40 - protein, 7 aa, Artificial Sequence
  Peptide Immunogen
GTNRPPV

SEQ ID NO: 41 - protein, 6 aa, Artificial Sequence
  Peptide Immunogen
TNRPPV

SEQ ID NO: 42 - protein, 5 aa, Artificial Sequence
  Peptide Immunogen
NRPPV

SEQ ID NO: 43 - protein, 17 aa, Artificial Sequence
  Peptide Immunogen
AATTTTYAYPGTNRPPV

SEQ ID NO: 44 - protein, 17 aa, Artificial Sequence
  Peptide Immunogen
TGTTTTYAYPGTNRPPV

SEQ ID NO: 45 - protein, 17 aa, Artificial Sequence
  Peptide Immunogen
TAATTTYAYPGTNRPPV

SEQ ID NO: 46 - protein, 17 aa, Artificial Sequence
  Peptide Immunogen
TATATTYAYPGTNRPPV

SEQ ID NO: 47 - protein, 17 aa, Artificial Sequence
  Peptide Immunogen
TATTATYAYPGTNRPPV

SEQ ID NO: 48 - protein, 17 aa, Artificial Sequence
  Peptide Immunogen
TATTTAYAYPGTNRPPV

SEQ ID NO: 49 - protein, 17 aa, Artificial Sequence
  Peptide Immunogen
TATTTTAAYPGTNRPPV

SEQ ID NO: 50 - protein, 17 aa, Artificial Sequence
  Peptide Immunogen
TATTTTYGYPGTNRPPV

SEQ ID NO: 51 - protein, 17 aa, Artificial Sequence
  Peptide Immunogen
TATTTTYAAPGTNRPPV

SEQ ID NO: 52 - protein, 17 aa, Artificial Sequence
  Peptide Immunogen
TATTTTYAYAGTNRPPV

SEQ ID NO: 53 - protein, 17 aa, Artificial Sequence
  Peptide Immunogen
TATTTTYAYPATNRPPV

SEQ ID NO: 54 - protein, 17 aa, Artificial Sequence
  Peptide Immunogen
TATTTTYAYPGANRPPV

SEQ ID NO: 55 - protein, 17 aa, Artificial Sequence
  Peptide Immunogen
TATTTTYAYPGTARPPV

SEQ ID NO: 56 - protein, 17 aa, Artificial Sequence
  Peptide Immunogen
TATTTTYAYPGTNAPPV

SEQ ID NO: 57 - protein, 17 aa, Artificial Sequence
  Peptide Immunogen
TATTTTYAYPGTNRAPV

SEQ ID NO: 58 - protein, 17 aa, Artificial Sequence
  Peptide Immunogen
TATTTTYAYPGTNRPAV

SEQ ID NO: 59 - protein, 17 aa, Artificial Sequence
  Peptide Immunogen
TATTTTYAYPGTNRPPA

SEQ ID NO: 60 - protein, 12 aa, Artificial Sequence
  Peptide Immunogen
TYAYPGTNRPPV

SEQ ID NO: 61 - protein, 13 aa, Artificial Sequence
  Peptide Immunogen
TTYAYPGTNRPPV

SEQ ID NO: 62 - protein, 14 aa, Artificial Sequence
  Peptide Immunogen
TTTYAYPGTNRPPV

SEQ ID NO: 63 - protein, 15 aa, Artificial Sequence
  Peptide Immunogen
TTTTYAYPGTNRPPV

SEQ ID NO: 64 - protein, 16 aa, Artificial Sequence
  Peptide Immunogen
ATTTTYAYPGTNRPPV